(19) United States
(12) Patent Application Publication
(10) Pub. No.: US 2006/0127946 A1
(43) Pub. Date: Jun. 15, 2006

(54) Reading of fluorescent arrays

(76) Inventors: Jean I. Montagu, Brookline, MA (US); Robert H. Webb, Lincoln, MA (US)

Correspondence Address: FISH & RICHARDSON PC, P.O. BOX 1022, MINNEAPOLIS, MN, 55440-1022, US

(21) Appl. No.: 10/524,615

(22) PCT Filed: Jan. 28, 2003
(86) PCT No.: PCT/US03/02502

§ 371 Date: Feb. 16, 2005

Related U.S. Application Data
(60) Provisional application No. 60/404,237, filed on Aug. 16, 2002.
(60) Provisional application No. 60/430,299, filed on Dec. 2, 2002.

(51) Int. Cl. G01N 33/53; C12M 1/34; G06K 7/00

(52) U.S. Cl. 435/7.1; 435/287.2; 235/435

(57) ABSTRACT

Reading of fluorescent arrays (103) in clinical settings is made possible by a reader (110) constructed to employ dark field illumination of the array, and mapping an image of the array onto a solid state sensor array (146) with image dimensions (D;) of the same order magnitude as the dimensions (D( ) of the fluorescent array, preferably with reduction of image. High intensity illumination is employed, non uniformities of which being compensated by normalization employing intensity calibration features (164) in the array itself, that are sensed during imaging of the array. Preferably high intensity light emitting diodes (122, 132, 402, 404), such as used in traffic lights, are employed for excitation of the array, preferably the excitation being introduced to the array via a solid internally reflecting homogenizer (130). Intermediate depth of field collection and imaging optics enable substantial collection of light, with NA in the range of 0.30 to 0.60, preferably in the range of 0.4 to 0.55. The resultant relatively large depth of field is in some advantageous cases compensated by absorbing light that tends to travel beyond the spots being imaged and would otherwise create noise fluorescence, the absorption produced e.g., by an opaque metal oxide coating (304) that is interposed between a substrate (302), preferably an ultra-thin substrate, on which the array lies, and the much thicker glass or other rigid support (306). For clinical purposes the arrays comprise fewer than 1000 spots, as is appropriate for protein, one example being an array of fewer than 500 spots. Relatively large spot sizes are employed, i.e. of the order of at least 80 or 100 micron diameter spots or preferably larger, 150 or 300 micron spots. Resolution of such spots to at least 50 pixels on the solid state detector array enables suitable binning and other manipulations leading to highly accurate results. Novel methods of assays and diagnosis such as cancer diagnosis employ the reader in detecting a set of markers related to the disease, for instance ovarian cancer.

### TECHNICAL FIELD

The field to which this disclosure relates is clinical micro-array technology, for instance clinical research and clinical diagnosis.

### BACKGROUND

Micro-array technology has developed over the past decade and more. It is employed in the investigation of biological molecules, in particular, nucleic acid and amino acid materials. Effective use has been made of the technology in understanding the genome and in drug discovery. It has been predicted that the technology would ultimately develop to enable practical use in the clinic, e.g. for clinical investigations and clinical diagnosis, but that prospect has seemed far off. Among reasons for this being only a long-term hope has been the very high cost of the required equipment, the time involved in carrying out assays, and the high level of experience and skill required.

As is well known, micro-arrays are used by creating a field of features or spots of different analytes that are tagged or marked if certain components are present. While marking has often been by radioisotope tags, fluorescent tags have come into wide usage for a number of reasons including the ease by which the materials can be handled and out of safety considerations. Typically it is desired to represent an assay by a complete image of an array, or small set of related arrays.

The reader of fluorescent micro-arrays is key to the use of the arrays. The reader records presence and degree of fluorescence at each of the precisely located features in the array in response to exposure to photoexcitation. After consideration of numerous arrangements for reader design, a few successful technologies have found acceptance. These universally have required precise and costly mechanical movements, as well as extensive optics or software. In one case, a rapidly oscillating scanner arm moves a tiny lens for reading one pixel at a time in one coordinate, in a confocal configuration, while the image of the array in the other coordinate is developed by precise, gradual advance using a microscope stage. (The stage is a device that creates precise movements of micron or sub micron accuracy and is costly to manufacture and along with the other components.) With this approach, software is used to assemble the image from the vast array of gathered pixels. Another technique has been to image highly magnified views of portions of the overall array, by use of precision stage movement between the taking of each of the series of magnified images of the small portions of the array, and then electronically merging or “stitching” the small field image frames together to electronically produce an image of the complete array. Prior proposals or speculation for employing a solid state array of sensors to image an entire array at one taking have not resulted in practical solution of the entire set of problems, i.e., simultaneously achieving high accuracy and high speed of operation at reasonable cost. As time has passed, and volume of production of readers has increased, the cost of those imaging systems that are successful, by elegant design, has been reduced from hundreds of thousands of today's U.S. dollars, in some instances to cost somewhat under one hundred thousand dollars. However, the prospect has seemed far off when volume production would enable the price for the readers to approach the cost that might make clinical usage attractive, e.g. a price of the order of twenty five thousand U.S. dollars or less.

### SUMMARY

In general, a reader capable of practical clinical use, i.e. in clinical research, clinical diagnosis and monitoring, is found to be possible with a certain combination while observing certain constraints, and it can be implemented with generally available components found in other fields. Embodiments can satisfy the crucial low cost need for a clinical reader, along with need for reasonably high speed and ease of operation, and while achieving the high level of accuracy required for medical use.

It is realized that the basic reader geometry should be based on dark field illumination, e.g. light reaching the two-dimensional array at an acute angle of 20 to 50 degrees, which should be mated with two-dimensional imaging on a solid-state detector array, such as that of a CCD sensor or CMOS array, with mapping on that array being of a scale of the same order of magnitude as the array of biological features. Imaging is performed at normal angle to the plane of the biological array, and is achieved with an optical collection and imaging system having an intermediate range numerical aperture (NA), preferably between NA=0.3 to 0.6, and presently, preferably within the range of NA=0.4 to 0.5.5. Embodiments within these constraints are capable of imaging an entire array in a single frame, without movement or stitching of components of the array. It is realized that cooperation of the features in the instrument, preferably with further novel enhancements to be described, can make up for the inherent limitations of such an arrangement, i.e. its relatively large depth of field, and, when using preferred relatively inexpensive lighting, such as by high intensity diodes, its non-uniform illumination. The resulting apparatus, because of its simplicity and lack of precise moving parts or expensive optics, can be made available to clinics at a cost that makes the system and technique practical. In such manner, practical, high-speed clinical imaging is made possible even now, and from this, great benefits to medicine and patient care can be obtained.

According to one aspect of the invention, an array reader is provided that is suitable for clinical purposes for reading a two-dimensional array of features on a planar substrate, in which the features carry photo-responsive markers, the markers capable of emitting light upon excitation, the array reader comprising an illumination system for simultaneously exciting multiple photo-responsive markers distributed in a two-dimensional array over the substrate, and an image collection and recording system having a field of view for emissions from the markers on the substrate, wherein the illumination system comprises a light source arranged to flood the two-dimensional array with light at an excitation wavelength, along an illumination path disposed at an angle θ between about 20 and 50° to the plane of the substrate, the image collection and recording system having an image-acquiring axis substantially normal to the plane of the substrate carrying the array, employing a two-dimensional sensor comprising a solid-state array of photosensitive elements, e.g. a charge-coupled device (CCD) or a CMOS array, and the image collection and recording system constructed and arranged to apply an image of the array of markers upon the solid-state array of size of the same order of magnitude as the size of the array, e.g. within a range of magnification of up to about 25% or reduction down to about 75%, the image collection and recording system having an intermediate numerical aperture NA, to enable recording the image of fluorescence from the excited two-dimensional array with clinical accuracy and without translation of the array.

Preferred embodiments of this aspect of the invention have one or more of the following technical features.

The array reader image collection and recording system has its nearest component spaced at least 5 mm, preferably at least 10 millimeter, from the substrate or its support, the component constructed and arranged to provide space below the component for the illumination path to the two-dimensional array on the substrate.

The image collection and recording system has an effective aperture between NA=0.3 and NA=0.60, preferably the value of NA being between about 4.0 and 5.5.

The image collection and recording system has a field of view on the substrate of areas between about 50 mm² and 300 mm².

The illumination system comprises one or more light-emitting diodes.

The illumination system, and especially the diode-based system, is constructed and arranged to provide excitation illumination over the two-dimensional array on the substrate of a power density greater than 30 mW/cm² and preferably the image collection and recording system includes a timer cooperatively related to the illumination system to provide exposure sufficient to produce a fluence of excitation radiation at the substrate greater than about 15 mJ/cm² across the two-dimensional array.

The array reader has a field of view of diameter of the order of 10 mm or more.

Each feature of the array of interest is imaged onto a minimum of 50 pixel elements of the solid state array, for example upon CCD or CMOS elements. (In the preferred case shown here the pixels (i.e. sensor elements) of the solid state array are selected to be of 9 micron dimension, albeit, if larger field were to be imaged, using the same arrangement, pixels down to about 4.5 micron may be selected, and more reduction of image may be employed).

The array reader is constructed and arranged to deliver to the solid state sensor an image of the field of view that is not magnified, preferably the reader being constructed and arranged to deliver to the solid state sensor array an image of the field of view reduced between about 30% and 50%.

The array reader is constructed to image spots each of diameter at least about 80 micron, preferably at least 100 micron diameter.

The array reader is constructed and arranged to produce, during a single imaging interval, an image of an array of at least 100 spots each of 300 micron diameter, or of at least 400 spots each of 150 micron diameter.

The array reader is combined with a carrier for the array comprising a substrate layer carried by a support body, the image collection and recording system residing on the same side of the substrate as does the array of features such that the path of illumination reaches the array before reaching the support body, the carrier constructed to absorb excitation radiation penetrating beyond the layer, preferably the support body being transparent, e.g. glass, and between the substrate layer and the transparent body resides a substantially opaque adherent layer capable of substantially blocking excitation radiation tending to enter the transparent body, preferably the substantially opaque layer comprising a layer of metal oxide.

The array reader is combined with a carrier for the array in the form of a transparent layer carried by a transparent body, the image collection and recording system lying beyond the transparent body on the same side of the array as the transparent body.

The array reader is combined with a carrier for the array that comprises an ultra-thin substrate layer on a support body, i.e. the substrate having a thickness less than 5 micron, preferably less than about 3 micron.

The array reader is combined with a carrier in which the array is disposed on a substrate comprising a clear layer of nitrocellulose or polystyrene.

The array reader is combined with a carrier in which the substrate is a nitrocellulose membrane that is porous at least in its outer region.

The array reader is combined with a substrate carrying excitation energy reference features distributed across the two-dimensional array of features, the image collection and recording system including a normalizing arrangement for normalizing data detected in the vicinity of respective reference features based on the quantity of detected emission from the respective reference features.

The array reader has an illumination system which comprises at least two different light source sub-systems respectively of substantially different wavelengths, each associated with a respective optical system delivering light along a path, the paths of the sub-systems to the substrate lying along respectively different axes, the axes being spaced apart about the substrate, in certain preferred embodiments there being two different light source subsystems the paths of which are disposed on diametrically opposite positions about the substrate.

The array reader has an illuminating system which includes light sources selected respectively to excite Cy3 and Cy5, and the image collection and recording system includes changeable band-pass filters suitable to permit passage of emissions respectively from Cy3 and Cy5 or a single band-pass filter is provided suitable to permit passage of multiple band-pass emissions such as both the band-pass emission of Cy3 and of Cy5.

The image collection and recording system of the array reader is adjustable between at least two settings, the first and second settings constructed and arranged respectively to form a single image of an array format of dimensions 6.5 mm×9.0 mm and of an array format comprising two separated sub-windows, each of dimensions 4.5 mm×4.5 mm disposed within a 4.5×13.5 mm rectangle.

The array reader illumination system includes a diode light source and a homogenizer effective to reduce variation in flux density across the field of illumination, in certain preferred embodiments the homogenizer comprising an elongated transparent, internally reflective rod, which may be straight or curved and may have round, square or rectangular cross section, and be twisted or untwisted.

The array reader has an image collection and recording system constructed and arranged to resolve the image on the solid state sensor array at resolution no finer than about 10 micron, in certain preferred embodiments the resolution being between about 12 and 15 micron.

The array reader has an image collection and recording system which includes an interference filter, collection optics of the system preceding the filter constructed to direct collected rays in parallel to the filter, and imaging optics constructed to image parallel rays leaving the filter upon the solid state sensor.

The array reader is constructed to be used with an array support that holds more than one array, and wherein the reader is constructed and arranged to read and process each array as an independent array.

The invention also includes a method of conducting an assay comprising preparing a two-dimensional spotted array of amino or nucleic acid features on a substrate, preferably by spotting liquid samples thereon, in which features in the array carry fluorescent markers and employing the reader of any of the foregoing descriptions to read the array.

Preferred embodiments of this aspect of the invention have one or more of the following technical features.

The assay is a diagnostic immuno assay based on protein derived from blood, in certain embodiments preferably the immunoassay is of an antibody capture configuration, for instance adapted, by immobilized antibodies to detect or monitor for malignant cancer, e.g. to detect ovarian cancer for initial diagnosis or to monitor patients at risk for relapse.

The substrate is disposed within a sealed disposable bio-cassette and imaging is performed through a transparent window visually accessing the substrate, or a transparent body forming a side of the bio-cassette carries the substrate, the substrate being transparent and the array being accessed visually by the array reader through the transparent body and through the substrate.

For any of the array reader embodiments described or for any of the foregoing methods, for reading an array on a substrate, in certain preferred embodiments the array includes intensity calibration markers of fluorescing character generally proportional in emission intensity to excitation level over the range of operable illumination intensities, and the system or method includes forming an image of the array employing the array reader, and normalizing recorded array data based on quantitative data acquired from nearby intensity calibration markers.

Another aspect of the invention is a fluorescence reader-based diagnostic method for a disease for which there is a set of known protein biomarkers in blood or other body constituent, comprising the steps of (1) providing a two-dimensional array of different reagents on a substrate, the reagents respectively specific to bind members of a set of the biomarkers capable of diagnosing the disease, (2) exposing the array to fluorophore-labeled blood or body-constituent extract of an individual containing the biomarkers if present in the individual's blood or body constituent, (3) while the array is stationary, exciting the array by simultaneously illuminating the entire two-dimensional array by light at fluorophore-excitation wavelength, by employing dark field illumination, (4) capturing a fluorescence image of the entire two-dimensional excited array on a single frame of an imager comprising a solid state array, and (5) analyzing (e.g. by computer) the fluorescence image for the presence of the disease.

Preferred embodiments of this aspect of the invention have one or more of the following features.

The method is performed in which the step of simultaneously illuminating the entire two-dimensional array is carried out by directing excitation radiation from a diode or set of diodes to produce illumination at a wavelength selected to excite the fluorophore, at a power density of at least 30 mW/cm².

The method is carried out in a way in which fluorescence intensity reference features are distributed through the array and the detected radiation from the bio-markers is normalized by the reader based on the response of the references to the illumination.

The method is carried out in a way in which at least 50 pixels of the solid-state sensor represent the image of a feature of the array.

The method is performed in which the biomarkers attach to antibodies.

The method is performed in a way in which the array is formed to immobilize protein biomarkers selected to diagnose presence of ovarian cancer.

Another aspect of the invention is a method of reading an array on a substrate having features that include fluorophores, in which the array includes intensity calibration features of fluorescing character generally proportional in emission intensity to their illumination over the range of operable illumination intensities, including, forming an image of the array employing an array reader, and normalizing recoded array data based on quantitative data acquired during the reading of the array from nearby intensity calibration features within the array.

Preferred embodiments of this aspect of the invention have one or more of the following features.

The method is adapted to perform diagnosis for a disease for which there is a set of known protein biomarkers in blood or other body constituent, comprising the steps of (1) providing a two-dimensional array of different reagents on a substrate, the reagents respectively specific to bind members of a set of the biomarkers capable of diagnosing the disease, and including with the array the intensity calibration features (2) exposing the array to fluorophore-labeled blood or body-constituent extract of an individual containing the biomarkers if present in the individual's blood or body constituent, (3) while the array is stationary, exciting the array by simultaneously illuminating the entire two-dimensional array by light at fluorophore-excitation wavelength employing dark field illumination, (4) capturing a fluorescence image of the entire two-dimensional excited array on a single frame of an imager comprising a solid state array, (5) normalizing the recorded array data based on the calibration features in the array and (6) analyzing the fluorescence image for the presence of the disease.

The method is performed by illuminating the entire two-dimensional array for forming the image by directing excitation radiation from a diode or set of diodes to produce illumination at a wavelength selected to excite the fluorophore, at a power density of at least 30 mW/cm².

The method is performed under conditions in which at least 50 pixels of a solid-state sensor represent the image of a feature of the array.

The method is employed to perform a diagnosis in which features of the array include antibodies, in one important case the features of the array are selected to diagnose the presence of ovarian cancer.

### DESCRIPTION OF DRAWINGS

FIG. 1 is a diagrammatic view of an array reading system.

FIGS. 2A and 2B are diagrammatic views of illuminating devices for the reader.

FIG. 3 is a plot of relative intensity of illumination versus angle relative to a central axis for a high intensity LED.

FIG. 4A depicts a spotting pin and reservoir suitable to form spots of biological material or intensity calibration spots, while FIG. 4B depicts an array in which the calibration spots are strategically distributed through the array of spots of biological material, FIG. 4C being a magnified view, and FIG. 4D a plan view.

FIG. 5 depicts the mapping of a spot upon the array of solid state detection elements of the sensor.

FIG. 6 is a diagrammatic representation, on highly magnified scale, of a carrier comprising a transparent rigid support body, bearing an opaque layer, ultra-thin substrate and spots of the array on the substrate. Illumination from the same side as the array is shown.

FIG. 7A is a diagrammatic representation of a preferred clinical array reader, while FIG. 7B shows another clinical array reader.

FIG. 8 illustrates two array formats imageable by the array reader of FIG. 7A.

FIG. 9 is a diagram representing the steps of an array-reading method.

FIG. 10 is a diagram representing the steps of a method for normalizing the intensity level of pixels in the recorded image.

FIG. 11 is a diagram representing the steps of a fluorescence reader-based diagnostic method.

Like reference symbols in the various drawings indicate like elements.

### DETAILED DESCRIPTION

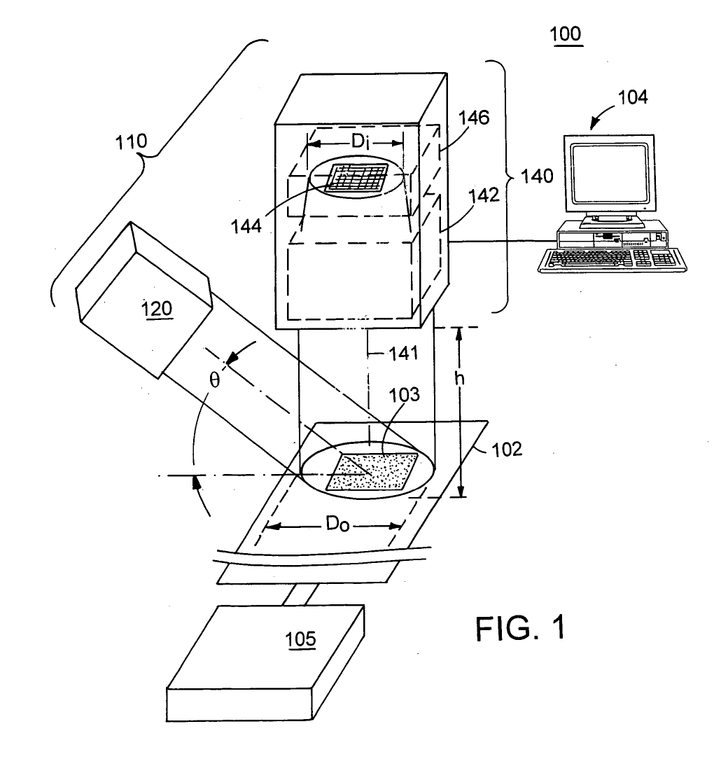

Referring to FIG. 1, an array reading system 100 includes an array reader 110, a substrate 102 bearing a two-dimensional array of features 103 (e.g. spots of bio-material such as amino or nucleic acid) some of which, depending on the assay, carry fluorescent material, and a computer 104 for processing images recorded with the array reader 110. The array reader 110 includes an illumination system 120 and an image collection and recording system 140.

During operation, the substrate 102 is positioned, with a positioner 105, below the image collection and recording system 140, with a distance between them h that is large enough for light from the illumination system 120 to flood the two dimensions of the array 103, preferably h having the value of at least 5 mm and generally preferably at least 10 mm. A preferred source of the illuminating light has an output between about 30 mW/cm² or more, and is preferably a light emitting diode (LED) or array of such diodes. The features on the substrate contain material capable of emitting light within a narrow fluorescence spectrum upon excitation with light of selected wavelength from the illumination system 120. Any available fluorescent dye may be employed, presently Cy3 and Cy5 being common selections.

The image collection and recording system 140 collects the fluorescent light and records a resulting image of the features or spots, the optical system selected to produce a flat field of view. The image-acquiring axis 141 is substantially normal to the plane of the substrate 102. The illumination system 120 uses dark field illumination such that light from the illumination system 120 is directed along a path that has an angle θ between about 20° and 50° to the plane of the substrate 102 to prevent illumination light reflected from the substrate 102 from entering the image collection and recording system 140. Fluorescent light emitted from the spots is collected by imaging optics 142 with an intermediate numerical aperture, e.g. between NA=0.30 to 0.60, and in presently preferred embodiments, in the range between NA=0.40 and 0.55. (all in air) to increase the field-of-view and the amount of light collected. The imaging optics 142 project an image 144 of the array 103 on a two-dimensional array of solid state detecting elements comprising sensor 146. This solid state array is of dimensions of the same order of magnitude as are the dimensions of the array of bio-material spots. The imaging optics system 142 is designed to have such a large field-of-view that the entire array 103 is mapped onto the sensor 146. Generally the two arrays are relatively sized such that a spot of bio-material is resolved on at least 50 pixels, preferably-with-spot size of the order of 100 micron, of the order of 100 pixels, or with spot size of 300 micron, of the order of 300 pixels.

With such construction, the physical size Dᵢ of the image 144 is approximately the same as the physical size Dₒ of the array 103, biased toward reduction, i.e. preferably not magnified more than about 25% or reduced more than about 75%. Presently, with reasonably low-cost available components, the array is not magnified, and preferably is reduced in the range between 30% and 50%. This provides the very important feature of there being no requirement to translate the array 103 by a precision stage relative to the reader 110 to acquire the image or stitch together multiple images of small sections to form a single image of the array 103.

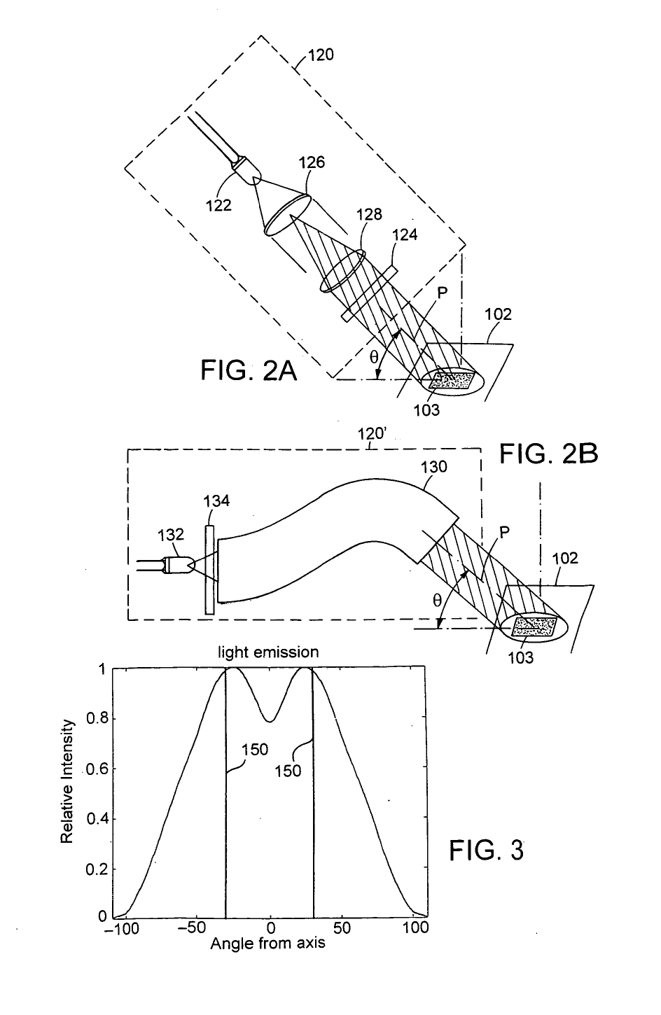

Referring to FIG. 2A, in a preferred embodiment, the illumination system 120 includes a high intensity LED 122 for emitting light within an excitation band (e.g., green) designed to excite a fluorescence spectrum of a material in a spot (e.g., Cy3). An excitation filter 124 is used to further limit the excitation wavelength band. The spatial distribution of the light is shaped with an optical system such as a pair of lenses 126 and 128 for near uniform illumination of the two-dimensional array 103 on the substrate 102. To provide higher intensity illumination, more than one LED can be distributed about the array 103, also arranged to emit light along a path that has an angle θ between about 20° and 50° to the plane of the substrate 102.

Referring to FIG. 2B, in another preferred embodiment, the illumination system 120′ includes a high intensity LED 132 for emitting light within an excitation band designed to excite a fluorescence spectrum of a material in a spot. An excitation filter 134 is used to further limit the excitation wavelength band. A homogenizer 130 with suitable lenses integral with its ends (not shown) reduces variation in flux density across the field of illumination onto the two-dimensional array 103 situated on the substrate 102. It comprises a solid transparent rod suitably designed or clad to have 100% internal reflection and of length relative to diameter selected to produce the desired homogenization effect, to render the distribution of illumination more uniform function. The homogenizer 130 accepts light up to an acceptance angle of approximately 45°. The end of the homogenizer 130 is arranged to emit light along a path that has an angle θ between about 20° and 50° to the plane of the substrate 102.

There are enhancements that can importantly be combined with the reader to raise performance of the reader to make it practical in various important contexts.

Using at least one, and preferably a distribution of energy calibration spots in each array, the detected intensity for spots of an array can be normalized against the detected value of radiation received from near-lying calibration spots during image processing, thus enabling tolerance of non-uniformity in radiation, as may occur in a low cost lighting system. The technique may also be effective to compensate for imprecise location of the array under the reader. FIG. 3 shows a non-uniform illumination pattern generated by a typical high-intensity LED, plotted as relative intensity as a function of angle from the center axis. The intensity of fluorescent light recorded by the solid state sensor array at a location in the image corresponding to a calibration spot is used to infer the local illumination intensity, which is used to normalize the signal level recorded at neighboring spots on the sensor array. Different calibrating spots typically local, respectively, to different sets of spots of unknown intensity are used.

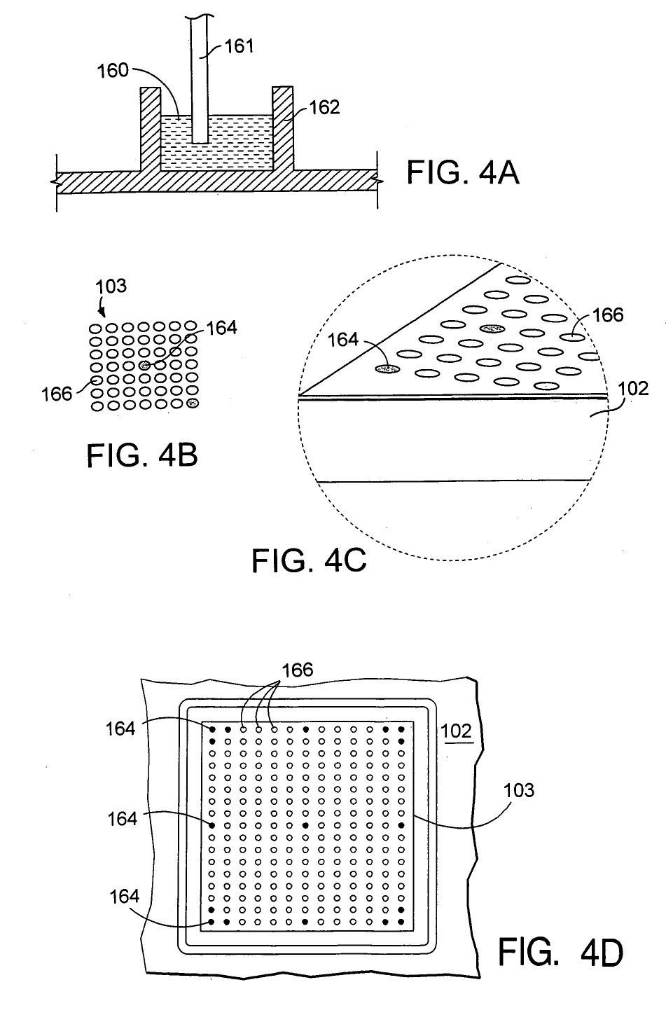

The calibration spots are preferably placed on the substrate along with the array of biological spots for accurate relative placement. FIG. 4A depicts a well of a microwell plate containing a fluorescent calibration composition in which a pin 161 is dipped to receive the composition for spotting a substrate 102 e.g. polyimide polymer (Kapton™) dissolved in a volatile solvent. FIG. 4B shows diagrammatically a spotted array 103 of biological spots 166 among which is a pattern of fluorescent calibration spots 164 produced with the composition of FIG. 4A. FIG. 4C is a magnified view of a portion of a substrate 102 containing, in addition to biological spots 166, fluorescent intensity calibration spots 164. FIG. 4D is a diagrammatic plan view, on an enlarged scale, of an array 103 of biological spots 166, showing a relative arrangement of calibration spots 164. This arrangement enables normalization of intensity variations across the entire array 103. The same intensity calibration spots can also be used as spatial fiducials for locating the array or the overall outline of the array may be employed for locating it to the control system.

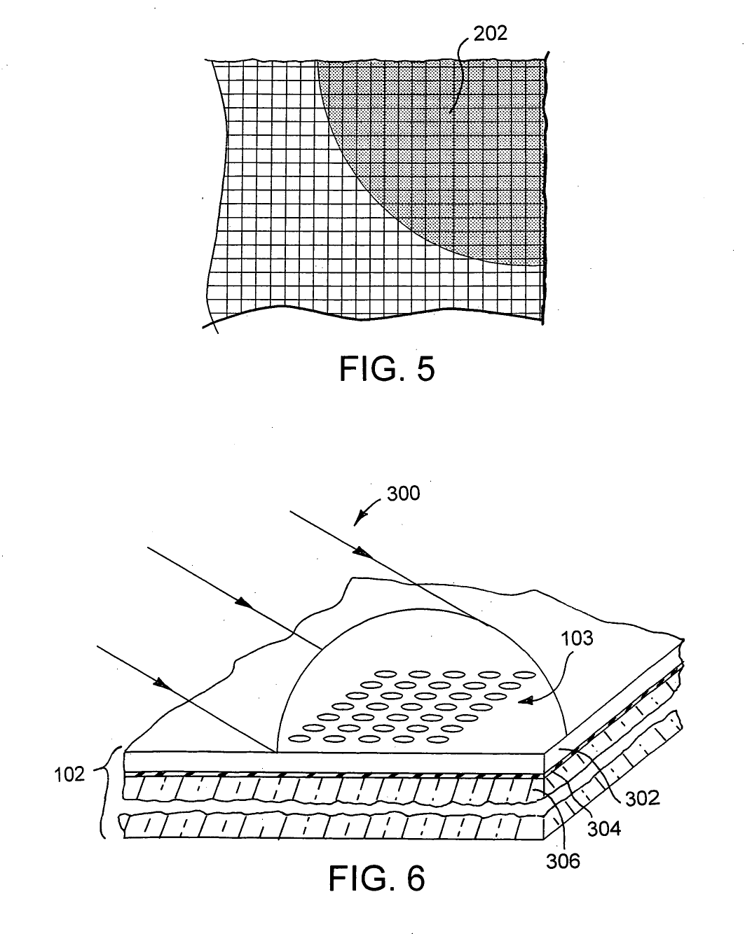

Taking advantage of the large size of the spots, hence the reasonable size of their image on the solid state array, despite no magnification, another enhancement is the binning of pixels in the solid state array image, which helps to average out random noise. The as-supplied on-board binning capability of a conventional CCD imager may thus be employed to enhance the accuracy of the reading. FIG. 5 shows a section of a CCD near a border of the image of a spot 202 corresponding enabling resolution of a feature in the array by approximately 291 pixels.

Other important enhancements involve provision of special features and characteristics of the substrate and its support which reduce auto-fluorescence capture (i.e., fluorescent light collected from sources other than the fluorescent material in the features of the array). FIG. 6 illustrates important aspects of a preferred substrate 102. An ultra-thin layer 302 of material (e.g., thinner than about 5 microns, preferably less than 3 microns) is comprised e.g. of nitrocellulose or polystyrene. As a film it is transparent, though in other cases an ultra-thin porous nitrocellulose membrane may be employed. The substrate supports the array 103 of features. The being ultra-thin, it limits the amount of fluorescence emitted by the substrate layer 302 itself.

An opaque layer 304, such as sputtered metal-oxide helps to prevent illumination light 300 from penetrating into, and exciting fluorescence within, the rigid support 306 below (e.g. the substance of a glass microscope slide). This helps to counter potential auto-fluorescence capture from the support layer 306 caused by the large depth of focus due to not using a high numerical aperture optical system.

The array reader 110 enables imaging of an array of fluorescently labeled proteins, as well as other potential widespread uses, such as imaging proteins labeled with luminescent tags, and with other bio-materials labeled with fluorescent or luminescent tags. The array reader 110 may be used to advantage with viruses, peptides, antibodies, receptors, and other proteins; with a wide range of other labeled biological materials including plant, animal, human, fungal and bacteria cells; and with labeled chemicals as well. The array reader 110 is designed for rapid imaging of immunoassay arrays of the size relevant to clinicians, with typically fewer than 1000 spots.

The array reader 110 also enables performing immunoassays of multiple biomarkers (e.g., for ovarian cancer) simultaneously. Diseases with a set of known protein biomarkers in blood or other body constituents, can therefore be diagnosed more easily. After providing a two-dimensional spotted array of reagents on a substrate, the reagent spots are exposed to fluorophore-labeled blood or other body-constituent extracted from an individual suspected of having the disease. The resulting array of spots are then read by the array reader 110.

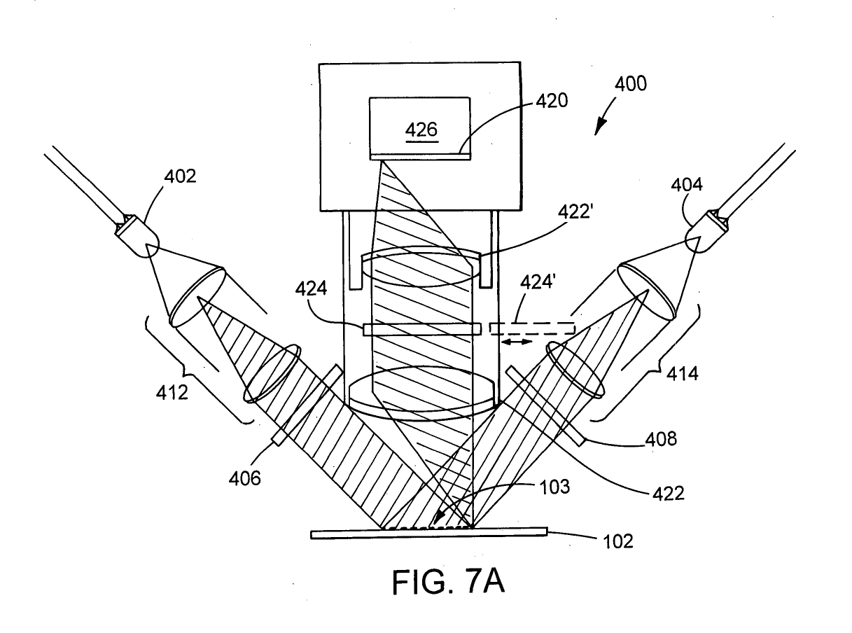

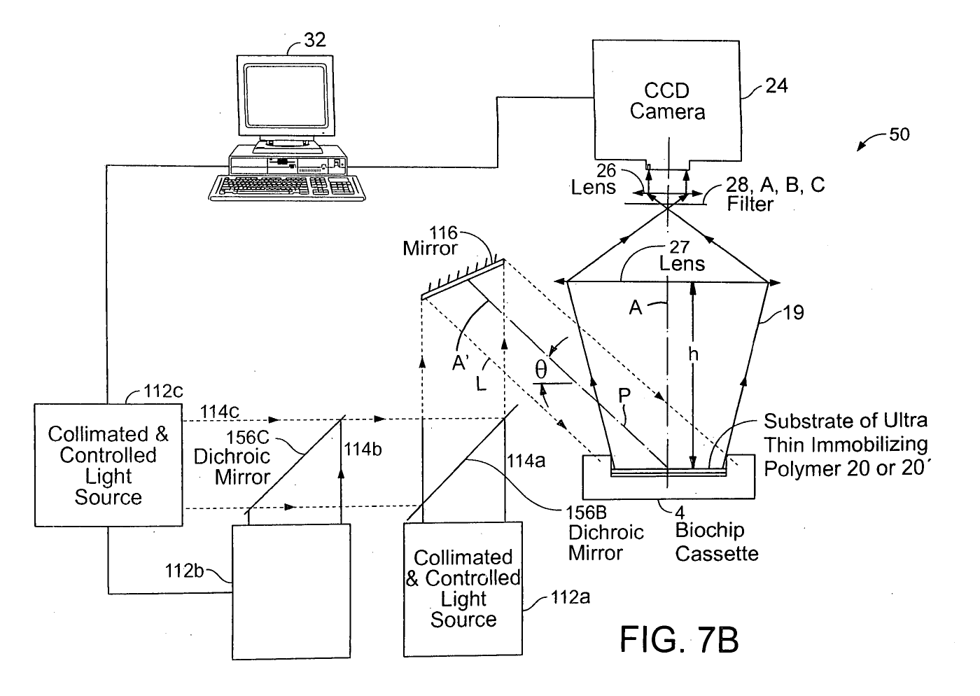

Referring to FIG. 7A, in a presently preferred embodiment two lighting sub-systems are provided at diametrically opposite positions about the array position, employing light sources originally designed for traffic lights. Array reader 400 includes a LumiLed high intensity LED 402 (Luxeon green 535 nm, 5 watt LED, part # LXHL-LM5C available from Lumileds Lighting U.S., LLC, San Jose, Calif.) with a green peak wavelength for excitation of Cy3, and a second LumiLed high intensity LED 404 (Luxeon red-orange 617 μm, 1 watt LED, part # LXH-MH1B) with a peak wavelength for excitation of Cy5. Both LEDs have a low temperature coefficient, of about 0.04 nm/deg C., and a narrow band peak wavelength tolerance, typically 8 nm for Cy5 and 30 nm for Cy3. These LEDs are available with a 10% to 20% conversion efficiency and a typical specification of 110 mW of continuous-wave output power that can be peaked by 50% for a second at low duty cycle to yield about 150 mW, nearly all within the pass bands of a Cy3 excitation filter 406 (Chroma filter part # HQ 535/50, available from Chroma Technologies, Rockingham, Vt.) and the Cy5 excitation filter 408 (Chroma filter part # HQ 620/60). The f/1 cone (marked by lines 150 at 30° in the illumination pattern shown in FIG. 3) includes 21.5% of the light, or 64.5 mW, (a larger capture is possible with the 45° acceptance angle of the homogenizer of FIG. 2B). However, all the light in the f/1 cone does not transfer through an f/1 lens because of Fresnel reflections at the higher angles. With proper filtering and vignetting it is safe to expect 50 mW (about 33% transfer efficiency) in a round beam 10 mm in diameter with less than 20% spatial intensity for the red LED 404. Tilting the beam by 45° spreads the light over an ellipse enclosing the desired 6.5×9.5 mm² area, so the power density is about 45 mW/cm². In ½ sec, that yields a fluence of 22.5 mJ/cm². For comparison, the excitation energy of a laser-confocal microscope is approximately 5 mW per 10 micron diameter spot for about 7.5 microsecond, yielding a fluence of 48 mJ/cm². Similar performances are obtained for the green LED 402 with peak absorption for Cy3. In addition to high power and cost efficiency, LEDS have a long life, and allow straightforward implementation of multi-color fluorescence. The green LED 402 uses a pair of Kohler lenses 412, and the red-orange LED 404 uses a pair of Kohler lenses 414, so that both LEDs deliver a nearly uniform beam over the 6.5×9.5 mm² field-of-view of the array 103. The LEDs are mounted on heat sinks available from their supplier.

Positioning of the substrate 102 relative to the viewing axis of the reader is performed by a positioner 105, e.g. a Geneva drive, with spatial resolution e.g. of 0.1 or 0.2 millimeter having a positional accuracy for instance of about 0.1-0.2 mm. The positioner 105 can be used to automatically shift from imaging one array to another, either on the same or a different substrate, but of course is not of the precision or cost of a microscope stage and plays no part in generating the components of an image of the array. The same substrate can carry many arrays without the need to precisely position the arrays relative to one another, and the positioner 105 acts to move one after another into position for imaging. Preferably, the substrate has alignment marks, “fiducials”, that aid in the positioning, for instance sets of distinctive marks that designate the corners of rectangular arrays.

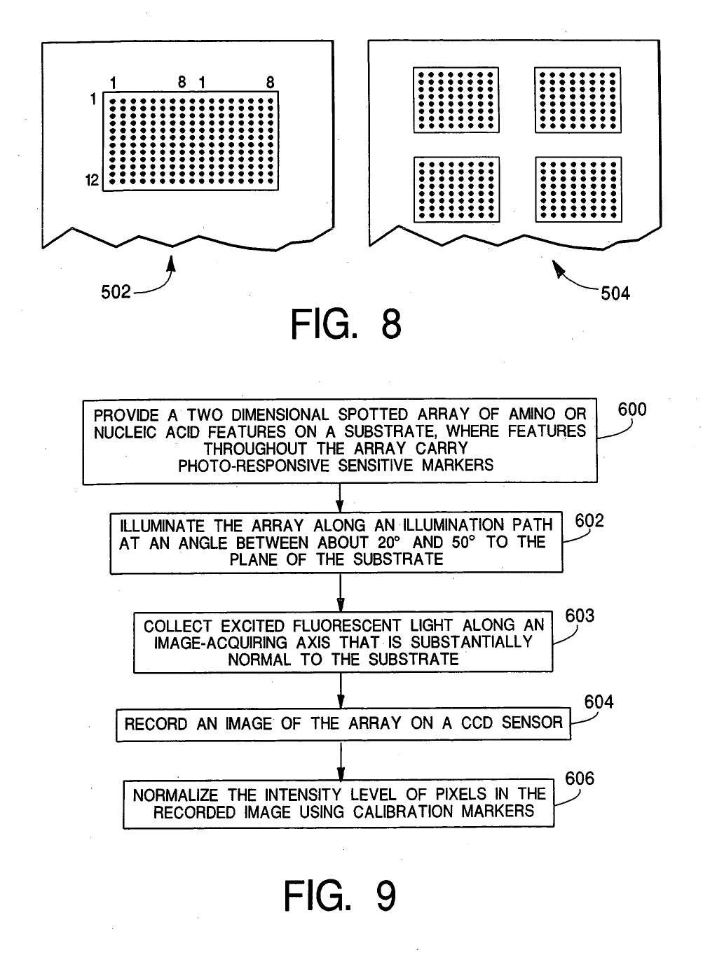

The array 103 is imaged onto the CCD sensor 420 by a pair of commercial CCD lens assemblies 422, 422′ (Westech CCD lens assemblies #2105 and #2131, available from Westech Optical Corporation, Penfield, N.Y.), lens 422 being used in an unusual way relative to the purpose of its original design. A band-pass filter 424 (Chroma Technology part # 68030 for Cy5, Chroma part # 57030 for Cy3) located in between the two lenses selectively transmits only light within the excited fluorescence spectra. The image of one 6.5×9.5 mm² field-of-view (see 502, FIG. 8) is projected onto the CCD sensor 420 reduced by a factor of 0.707, whereas the image of two separated sub-arrays, lying in a rectangle 4.50×13.5 mm is reduced by a factor of 0.5.

The lenses are assembled to operate with a 0.42 NA on the object side (facing the array 103). The array 103 can be imaged onto the CCD sensor 420 with the lens assemblies 422 assembled to operate with an NA as large as 0.52.

The CCD sensor 420 is cooled with a Peltier cooler 426 (as in the CCD-based camera from Santa Barbara Instrument Group, Inc., Santa Barbara, Calif., Model ST-7×ME) to reduce dark current noise. The cooler 426 has the capability to cool the CCD sensor 420 to 50° C. below ambient if necessary. Despite the advantageous cooling, read-out noise, generated upon conversion of the stored charge in a pixel into a voltage, is a dominant source of noise and to the extent its effects are not eliminated, read-out noise determines the minimum light intensity that can be detected. As this noise is random and the fluorescent light from the spots is not, most of its effects can be reduced by the “on board binning,” dark field subtraction, time and frame integration, software analysis, and using a large number of pixels imaging each spot.

Referring to FIG. 7B, imaging in a dark field mode may also be accomplished with direct illumination at angle θ as shown and CCD sensor 24 positioned to view the array along axis A normal to the plane of the array via collection optics 27, spaced a distance h from the substrate. In this case the substrate layer may be microporous partially or throughout its depth or may be a solid film or a modified solid film, preferably in any of these cases being an ultra-thin coating or membrane of less than 5 micron thickness. As shown, light for direct illumination enters along an illumination axis A′, at an acute angle θ to the plane of the array. Distance h must be selected to enable such direct illumination, with angle θ ranging between about 200 and 50°, here shown at 45°. Light L originates from a source 112a, 112b or 112c of wavelength selected to excite the fluorophore tag of the array, passes via dichroic mirrors 156b, 156c to mirror 116 located to the side that directs the illumination along axis A′ at angle θ, onto the fluorophore-tagged array of spots resident on the ultra-thin substrate 20 or 20′. The array of spots may use a carrier that comprises an ultra-thin substrate layer on a support body, or a carrier in the form of a transparent layer carried by a transparent body, the image collection and recording system lying beyond the transparent body on the same side of the array as the transparent body. The fluorescent emissions are collected by lens 27, through a selected filter 28A, B or C, thence through lens 26 to CCD camera 24 under computer control 32. As before, the background subtraction technique is used with this system. The differences between the excitation source and that of FIG. 7A, come at a significant cost that is counter to the most cost-demanding situations of the clinic, but the geometry and different capabilities of the lighting system of FIG. 7B can have advantages that enjoys the other benefits that have been described.

In an advantageous design, the immunoassay arrays are limited to 400 microassay spots. The array format is then an important design issue. The size and number of pixels of the CCD's chip and the configurations of commercial spotting arrayers are important constraints to be balanced against each other in the design of the reader and array. The size of the array must be matched to the CCD's parameters. On the other hand, spotter pin or tip configurations limit the choice of reservoirs for loading the spotting or printing head with source material, and also limit possible array configurations. Disposable microtiter plates with either 96 or 384 wells are the typical reservoirs used. These constraints are satisfied by the two formats presented schematically in FIG. 8.

Taking into consideration geometric tolerances, a first format 502 field-of-view covers one 6.5×9.5 mm² array, and a second format 504 field-of-view covers two 4.5×4.5 mm² arrays. Assuming 300-micron diameter spots, 500 microns on center, yielding a spot occupancy of 36%, each spot will be conjugated to about 291 pixels. Assuming 150-micron diameter spots, 333 microns on center, each spot will be conjugated to about 91 pixels. The large number of pixels per spot permits the on board 3×3 binning option available with the CCD sensor to increase signal-to-noise ratio. The immediate background is subjected to the same averaging to yield a sensitive and reliable fluorescence signal level. Arrays can be formed with each of the formats using either of the two spot sizes. These arrays can be printed with all commercial arrayers/printers, such as the Affymetrix Pin and Ring Arrayer, starting with either 96 or 384 microtiter plates as the source material loading reservoir.

Other methods are useful to raise the signal-to-noise ratio to best define the quality of the image. Longer integration time or the sum of multiple acquisitions of the stationary array are useful to avoid CCD saturation. The signal-to-noise ratio improves as the square root of the ratio of integration time or the number of frames. A 5 second read time versus 0.5 seconds improves the signal-to-noise ratio by approximately 3.16 times.

The signal-to-noise ratio can also be increased by increasing the number of LEDs, e.g. to as many as 4 for each of the 2 wavelengths, to raise the power level to 160 mW/cm2. Applied together, these options increase the signal to noise ratio by as much as a factor of 13 by substantially raising the fluence to 1,120 mJ/cm². Photo-bleaching, a possible consequence, depending upon the dyes etc., may limit this approach in particular circumstances.

Alternate embodiments can use other types of substrates. For example, the substrate can be a glass slide, or alternatively, a sealed disposable bio-cassette where imaging is performed through a transparent window within the substrate.

Referring to FIG. 9, a method for multi-biomarker assay includes the step 600 of providing a two-dimensional spotted array of amino or nucleic acid features on a substrate, where features throughout the array carry photo-responsive sensitive markers. In the second step, 602, the illumination source of the array reader illuminates the array along an illumination path at an angle θ between about 20 and 50° to the plane of the substrate. In the third step, 603, the image collection and recording system then collects excited fluorescent light along an image-acquiring axis that is substantially normal to the substrate, followed by the step 604 of recording an image of the array of bio-material spots on the solid array of a CCD sensor, followed by the step 606 of normalizing the intensity level of pixels in the recorded image using intensity calibration markers.

It is to be noted that this calibration occurs as an integrated action in the imaging of each array. It is to be distinguished from pre-reading calibration of the overall instrument, a normal but not totally effective procedure.

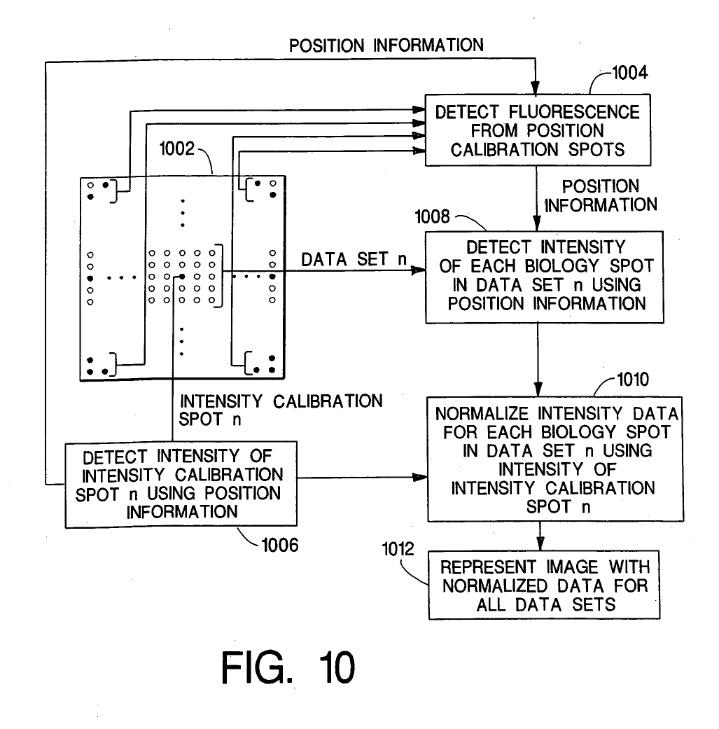

Referring to FIG. 10, a method for normalizing the intensity level of pixels in the recorded image includes the step 1004 of determining pixels that detect fluorescence from position calibration spots located at corners of an array 1002. The resulting position information is then used to locate pixels that correspond to multiple “data sets” across the two-dimensional image. Each data set contains pixels corresponding to biology spots, and pixels corresponding to an intensity calibration spot. For each data set, including a “data set n,” the method includes the step 1006 of detecting intensity recorded by pixels representing the intensity calibration spot n, the step 1008 of detecting intensity recorded by pixels representing each biology spot in data set n, and the step 1010 of normalizing the intensity data for each biology spot using the intensity of the intensity calibration spot. After intensity data is normalized for each data set, in a final step 1012, the entire image is represented according to the normalized data for all of the data sets.

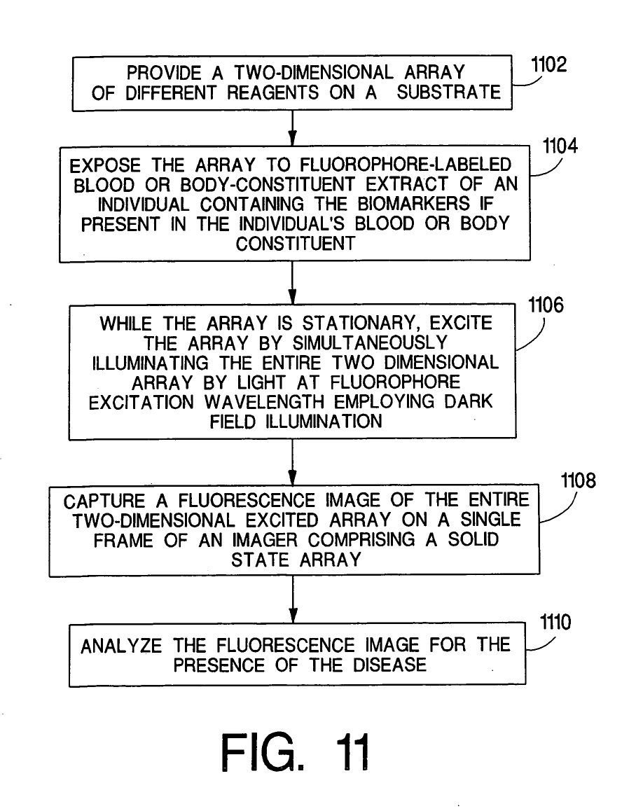

Referring to FIG. 11, a fluorescence reader-based diagnostic method, for a disease for which there is a set of known protein biomarkers in blood or other body constituent, includes the step 1102 of providing a two-dimensional array of different reagents on a substrate. The reagents are respectively specific to bind members of a set of the biomarkers capable of diagnosing the disease. The method then includes the second step 1104 of exposing the array to fluorophore-labeled blood or body-constituent extract of an individual containing the biomarkers if present in the individual's blood or body constituent. While the array is stationary, in a third step 1106, the reader excites the array by simultaneously illuminating the entire two-dimensional array by light at a fluorophore excitation wavelength, employing dark field illumination. In a fourth step 1108, the reader then captures a fluorescence image of the entire two-dimensional excited array on a single frame of an imager comprising a solid state array. The method then includes the step 1110 of analyzing the fluorescence image for the presence of the disease.

In a preferred embodiment the assay is a diagnostic immunoassay based on protein derived from blood, and can detect or monitor for malignant cancer, such as ovarian cancer, for initial diagnosis or to monitor patients at risk for relapse. In the last decade, the search for biomarkers that alone, or in combinations with Ca125, could improve prognostic testing for ovarian cancer yielded a number of candidates. However, in 1995, Berek and Bast reviewed data on 17 different markers (including CA125) and concluded that none was useful in the setting of early stage ovarian cancer (1). However, it has been shown that other tumor markers can complement CA125 and be useful in some circumstances (2).

Recently, genomic technologies have dramatically accelerated progress in the search for prognostic ovarian cancer biomarkers. Studies using differential DNA transcriptional profiling of ovarian cancer cell lines and those from ovarian epithelium collectively have identified hundreds of candidate biomarkers (e.g. 3, 4, 5). Some of these new candidate protein biomarkers have been evaluated in exploratory trials. Candidate biomarker proteins which have been studied include: HE4 (6), osteopontin (7), prostasin (4, 5) and mesothelin/megakaryocyte potentiating factor (8). Recent reports also suggest that members of the kallikrein serine protease family, particularly kallikrein 10, may also serve as ovarian cancer biomarkers in blood (9, 10, 11). Results from exploratory studies are encouraging and suggest that these proteins either alone, or in combinations with other markers such as CA125, may be useful as prognostic indicators for ovarian cancer.

There is direct evidence that patterns of multiple biomarkers in blood provide a signal of early stage ovarian cancer. Proteomic spectra generated by mass spectroscopy (SELDI-TOF) from sera of ovarian cancer patients and normal individuals and analyzed by an iterative searching algorithm, identified a proteomic profile of five, of as yet unidentified proteins, that completely discriminated the sera of the ovarian cancer patients (12). In a test of blinded samples, the discriminatory pattern correctly identified 100% of the ovarian cancer samples including the 36% from early stage patients and showed a specificity (false positive rate) of 95% (12).

If the positive predictive value of proteomic pattern technology is supported by population-based trials, these discriminating proteins provide excellent opportunities for developing highly sensitive diagnostic probes (13) which can, given the appropriate technology platforms, be ultimately exploited in routine tests for detecting early stage ovarian cancer.

The references referred to are:

1. Berek, J S, R C Bast. 1995 Ovarian cancer screening. The use of serial complementary tumor markers to improve sensitivity and specificity for early detection. Cancer 76 2092-06.

2. Woolas, R P, D H Oran, A R Jeyarajah, R C Bast, J J Jacobs, 1999. Ovarian cancer identified through screening with serum markers but not by pelvic imaging. 1999. Int. J. Gynecol 9: 497-501.

3. Schummer, M, W V Ng, R E Baumgarner, PS Nelson, B Schummer, D W Bednarski, L Hassell, B Y Baldwin and L. Hood. 1999. Comparative hybridization of an array of 21,500 ovarian cDNAs for the discovery of genes over-expressed in ovarian carcinoma Gene 238: 375-385.

4. Kwong-Kwok, W, R S Cheng, S C Mok. 2001. Identification of differentially expressed genes from ovarian cancer cells by MICROMAX™ cDNA microarray system. 2001. Biotechniques 30(3): 670-674.

5. Mok, S, J Chao, S Skates, K-K Wong, G K Yu, M G Muto, RS Berkowitz, D W Cramer. 2001. Prostasin, a potential Serum Marker for Ovarian Cancer: Identification Through Microarray Technology. JNCI 93 (19): 1458-1464.

6. Hellstrom, I, J. Raycaft, M Hayden-Ledbetter, J A Ledbetter, M Schummer, M McIntosh, C. Drescher, N Urban, K E Hellstrom. 2003. The HE4 (WFDC2)-protein is a biomarker for ovarian carcinoma. Cancer Research 63; 3695-3700.

7. Kim, J H, S J Skates, T Ude, K K Wong, J O Schorge, C M Feltmate, RS Berkowitz, D W Cramer, S C Mok. 2002. Osteopontin as a potential diagnostic biomarker for ovarian cancer. JAMA 287 (13):1671-9.

8. Scholler N, N Fu, Y Yang, Z Ye, G E Goodman, K E Hellstrom, I Hellstrom. 1999. Soluble member(s) of the mesothelin/megakaryocyte potentiating factor family are detectable in sera from patients with ovarian carcinoma. PNAS USA 96 (20): 11531-6. (found in the L. Hood screening study)

9. Shvartsman, H S, K H Lu, J Lillie, M T Deavers, S Clifford, J I Wolf, G B Mills, R C Bast, D M Gershenson, R Schmandt. 2003. Over expression of kallikrien 10 in epithelial ovarian carcinomas. Gynecol Oncol 90: 44-50.

10. Luo, L Y, D Katsaros, A Sorilas, S Fracchiolo, R. Riccinno, I A Rigault de la Longris, D J C Howarth, E P Diamandis 2001. Prognostic value of human kallikrein 10 expression in epithelial ovarian carcinoma Clin Can Res 7: 2317-2379.

11. Luo, L Y, D Katsaros, A Scorialas, S Fracchioli, R Bellino, M van Gramberen, H de Bruijn, A. Henrik, UH Stenman, M Massobrio, A G van der Zee, I Vergote, EP Diamandis. 2003. The serumprognosis. Cancer Res 63: 807-11.

12. Petricoin, E F, A M Ardekani, B A Hitt, P J Levine, V A Fusaro, M A Steinberg, G B Mills, C Simone, D A Fishman, E C Kohn, L A Liotta 2002. Use of proteomic patterns in serum to identify ovarian cancer. Lancet 16: 359 (9306);572-77.

13. Wulfkuhle, J D, L A Liotta, E F Petricoin. 2003. Proteomic applications for the early detection of cancer. Nature Reviews Cancer 3: 267-75.

For further disclosure concerning the topics of (1) employing the characteristics of ultra-thin substrate layers in dark field illumination and imaging on a solid state array of sensors of size of order of magnitude of the array of spots, in general and in particular of nitrocellulose and polystyrene, and their methods of manufacture and use, (2) metal oxide and other absorbent layers beneath the substrate that absorb excitation light serving to enhance the operation or make practical a clinical fluorescence reader and (3) formation and utilization of intensity calibration marks in micro-arrays for serving to enhance the operation of a fluorescence reader, in particular one using a high intensity light emitting diode or diode array for excitation illumination, reference is made to a further PCT application being filed simultaneously herewith, which likewise claims priority from U.S. Provisional Ser. No. 60/476,512, filed Jun. 6, 2003.

Other features and advantages of the invention will be understood from the foregoing and the claims and are within the spirit and scope of the invention.

1. An array reader suitable for clinical purposes for reading a two-dimensional array of features on a planar substrate, in which the features carry photo-responsive markers, the markers capable of emitting light upon excitation, the array reader comprising:
an illumination system for simultaneously exciting multiple photo-responsive markers distributed in a two-dimensional array over the substrate,
and an image collection and recording system having a field of view for emissions from the features on the substrate,
wherein the illumination system comprises a light source in the form of at least one light-emitting diode arranged to flood the two-dimensional array with light at an excitation wavelength, along an illumination path disposed at an angle (θ) between about 20° and 50° to the plane of the substrate,
the image collection and recording system having an image-acquiring axis substantially normal to the plane of the substrate carrying the array, employing a two-dimensional sensor comprising a solid-state array of photosensitive elements, and the image collection and recording system constructed and arranged to apply an image of the array of features upon the solid-state array of size of the same order of magnitude as the size of the array, e.g. within a range of magnification of up to about 25% or reduction down to 75%, the image collection and recording system having an intermediate numerical aperture NA to enable recording the image of fluorescence from the excited two-dimensional array with clinical accuracy and without translation of the array.

2. (canceled)

3. The array reader of claim 1 in which the image collection and recording system has an effective aperture between NA=0.3 and NA=0.60.

4-5. (canceled)

6. The illumination system of claim 1 constructed and arranged to provide excitation illumination over the two-dimensional array on the substrate of a power density greater than 30 mW/cm².

7. (canceled)

8. The array reader of claim 1 in which the field of view of the array reader has a diameter of the order of 10 mm or more.

9. (canceled)

10. The array reader of claim 1 constructed and arranged to deliver to said solid state sensor array an image of the field of view that is not magnified.

11. The array reader of claim 1 constructed and arranged to deliver to said solid state sensor array an image of the field of view reduced between about 30% and 50%.

12-13. (canceled)

14. The array reader of claim 1 in combination with a carrier for the array comprising a substrate layer carried by a support body, said image collection and recording system residing on the same side of the substrate as does the array of features such that the path of said illumination reaches said array before reaching the support body, said carrier constructed to absorb excitation radiation penetrating beyond said layer.

15. The array reader of claim 14 in which said support body is transparent, and between said substrate layer and said transparent body resides a substantially opaque adherent layer capable of substantially blocking excitation radiation tending to enter the transparent body.

16. The array reader of claim 15 in which said substantially opaque layer comprises a layer of metal oxide.

17. The array reader of claim 1 in which said substrate is in the form of a transparent layer carried by a transparent body, the image collection and recording system lying beyond the transparent body on the same side of the array as the transparent body.

18. The array reader of claim 1 in combination with a carrier for said array that comprises a substrate layer on a support body, the substrate having a thickness less than about 5 micron.

19. The array reader of claim 1 in which said array is disposed on a substrate comprising a clear layer of nitrocellulose.

20. (canceled)

21. The array reader of claim 1 in combination with a substrate carrying excitation energy reference features distributed across said two-dimensional array of features, said image collection and recording system including a normalizing arrangement for normalizing data detected in the vicinity of respective reference features based on the quantity of detected emission from the respective reference features.

22. The array reader of claim 1 in which said illumination system comprises at least two different light source sub-systems respectively of substantially different wavelengths, each associated with a respective optical system delivering light along a path, the paths of said sub-systems to said substrate lying along respectively different axes, the axes being spaced apart about said substrate.

23. (canceled)

24. The array reader of claim 1 in which said illuminating system includes light source diodes selected respectively to excite Cy3 and Cy5, and said image collection and recording system includes changeable band-pass filters suitable to permit passage of emissions respectively from Cy3 and Cy5 or a single band-pass filter is provided suitable to permit multiple band-pass emissions of Cy3 and Cy5.

25. (canceled)

26. The array reader of claim 1 in which said illumination system includes a diode light source followed by a homogenizer effective to reduce variation in flux density across the field of illumination.

27-38. (canceled)

39. A fluorescence reader-based diagnostic method for a disease for which there is a set of known protein biomarkers in blood or other body constituent, comprising the steps of (1) providing a two-dimensional array of different reagents on a substrate, the reagents respectively specific to bind members of a set of said biomarkers capable of diagnosing the disease, (2) exposing the array to fluorophore-labeled blood or body-constituent extract of an individual containing the biomarkers if present in the individual's blood or body constituent, (3) while the array is stationary, exciting the array by simultaneously illuminating the entire two-dimensional array by light at fluorophore-excitation wavelength employing dark field illumination, (4) capturing a fluorescence image of the entire two-dimensional excited array on a single frame of an imager comprising a solid state array, and (5) analyzing the fluorescence image for the presence of the disease.

40. (canceled)

41. The method of claim 39 in which fluorescence intensity reference features are distributed through the array and the detected radiation from said bio-markers is normalized by the reader based on the response of said references to said illumination.

42-44. (canceled)

45. A method of reading an array on a substrate having features that include fluorophores, in which the array includes intensity calibration features of fluorescing character generally proportional in emission intensity to their illumination over the range of operable illumination intensities, including, forming an image of the array employing an array reader, and normalizing recorded array data during the reading of the array from nearby intensity calibration features within the array.

46-50. (canceled)
